(19) United States
(12) Patent Application Publication
(10) Pub. No.: US 2022/0041905 A1
(43) Pub. Date: Feb. 10, 2022

(54) GREEN POLYETHYLENE WAX FOR HOT MELT ADHESIVES, COATINGS, AND COSMETICS

(71) Applicant: Braskem S.A., Camaçari (BR)

(72) Inventors: Suzana Kupidlowski Boaventura, São Paulo (BR); Dhananjay Ramesh Patil, Rotterdam (NL); Rafael Pedro Pellicciotta, São Paulo (BR)

(73) Assignee: Braskem S.A., Camaçari (BR)

(21) Appl. No.: 17/396,292

(22) Filed: Aug. 6, 2021

Related U.S. Application Data
(60) Provisional application No. 63/061,993, filed on Aug. 6, 2020.

(51) Int. Cl. C09J 123/06; C09J 129/04; A61K 8/81; A61Q 19/00; C08L 23/06

(52) U.S. Cl. CPC C09J 123/06; C09J 129/04; C08L 23/06; A61K 8/8129; A61Q 19/00; A61K 8/8111

(57) ABSTRACT

A composition may include a polyethylene wax, in which the polyethylene comprises at least a portion of ethylene that is obtained from a renewable source of carbon. Compositions may be, and articles may include, a hot melt adhesive composition, a filler masterbatch composition, a cosmetic composition, an ink composition or a PVC processing composition.

### BACKGROUND

Polyolefins such as polyethylene (PE) are widely used worldwide, given their versatility in a wide range of applications, including the manufacture of films, coatings, adhesives, molded products, and the like. The increasing complexity of manufactured goods has led to major improvements and developments in many of these applications. In particular, synthetic waxes (such as PE waxes) are widely used in a variety of compositions as they may impart unique properties. PE waxes may, for instance, provide lubrication to, or modify the viscosity or melt point of, a composition.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a composition that includes a polyethylene wax, in which the polyethylene comprises at least a portion of ethylene that is obtained from a renewable source of carbon.

In another aspect, embodiments disclosed herein relate to an article that includes a substrate and a coating on the substrate, where the coating includes to a composition that includes a polyethylene wax, in which the polyethylene comprises at least a portion of ethylene that is obtained from a renewable source of carbon.

In another aspect, embodiments disclosed herein relate to an article that includes a composition that includes a polyethylene wax, in which the polyethylene comprises at least a portion of ethylene that is obtained from a renewable source of carbon.

In yet another aspect, embodiments disclosed herein relate to a process for producing a composition that comprises a polyethylene wax where the method includes combining the polyethylene wax with at least one other component to form the composition, in which the polyethylene comprises at least a portion of ethylene that is obtained from a renewable source of carbon.

Other aspects and advantages of the claimed subject matter will be apparent from the following description and the appended claims.

### BRIEF DESCRIPTION OF DRAWINGS

FIG. 1 shows a change in rheology behavior of adhesives as a function of amount of wax added.

### DETAILED DESCRIPTION

One or more embodiments disclosed herein relate to polymer compositions that comprise a green polyethylene (PE) wax. The green PE wax may contain a portion of biobased polyethylene that is derived from a renewable source of carbon, such as a plant-based material. In one or more embodiments, the polymer compositions may be formulated for many different applications. In some embodiments, the polymer compositions may be used as hot melt adhesives, masterbatches, coatings, inks and paints, plastic additives, and the like. One or more embodiments of the present disclosure are also directed to articles that comprise a polymer composition. Other embodiments are directed to processes for creating articles that include a polymer composition.

Polymer compositions in accordance with the present disclosure may be formulated with at least part of a fraction of biobased polyethylene (PE) as a replacement for (or in addition to) PE derived from petrochemical sources. As used herein, “biobased PE” is a PE wherein the ethylene constituting the polymer is derived from renewable sources, such as biobased ethanol.

The use of materials derived from renewable sources are preferred to those obtained from fossil sources in order to reduce carbon dioxide emissions and mitigate the impact of polymer production on climate change. Polymer compositions in accordance with the present disclosure may reduce the overall impact on carbon dioxide levels, relative to conventional compositions, by incorporating at least a portion of materials that are obtained from renewable carbon sources. This renewable carbon content can be certified by the methodology described in the technical standard ASTM D6866-18, “Standard Test Methods for Determining the Biobased Content of Solid, Liquid, and Gaseous Samples Using Radiocarbon Analysis.” in contrast to petroleum-sourced compositions, compositions obtained from renewable natural raw materials may be incinerated at the end of their life cycle and only producing CO₂ of a non-fossil origin.

### Green Polyethylene Waxes

One or more embodiments in accordance with the present disclosure are directed to polymer compositions containing a “green PE wax,” i.e. a polyethylene wax where at least a portion of the ethylene that constitutes the PE wax is obtained from a renewable source of carbon. In some embodiments the ethylene obtained from a renewable source of carbon may be biobased, such as that derived from a plant-based material.

The PE wax of one or more embodiments may comprise a homopolymer of ethylene. In some embodiments, the PE wax may comprise a copolymer resulting from the polymerization of ethylene and one or more comonomers. Such comonomers may include, for example, acrylic acid, vinyl acetate, and maleic anhydride. In embodiments where the PE wax comprises a copolymer, one or more of the ethylene and the comonomers may be derived from renewable sources. Such comonomers may be present in an amount up to 40 wt %.

The PE wax of one or more embodiments can be made by direct polymerization of ethylene under conditions to control molecular weight and branching of the polymer. However, in particular embodiments, a high molecular weight PE resin may be cracked into lower molecular weight fractions to yield the PE wax. Another method of other embodiments may include the separation of a low molecular weight fraction from a production stream of high molecular weight PE.

The PE wax of one or more embodiments may be produced by the thermal degradation and/or depolymerization of PE. As discussed above, in some embodiments at least a portion of the PE may be bio-based PE that has a carbon content derived from a renewable source. In some embodiments, the PE used to produce the PE wax is not particularly limited, and may be selected in order to provide a PE wax having properties suitable for its intended application.

In one or more embodiments, the PE used to produce the PE wax may have a melt flow rate (MFR), measured according to ASTM D1238 at 190° C. and 2.16 kg, ranging from 0.1 to 40 g/10 min. In one or more embodiments, the PE may have a MFR, measured according to ASTM D1238 at 190° C. and 2.16 kg, of a range having a lower limit of any of 0.1, 1, 5, 10, 20, 24, 30, and 32 g/10 min, and an upper limit of any of 20, 24, 28, 32, 34, 36, 38, or 40 g/10 min, where any lower limit can be used in combination with any upper limit.

In one or more embodiments, the PE used to produce the PE wax may have a bio-based carbon content, as determined by ASTM D6866-18 Method B, of at least 5%. Further, other embodiments may include at least 10%, 20%, 40%, 50%, 60%, 80%, 90%, 95%, 99%, or 100% bio-based carbon. As discussed above, in embodiments where the PE used to produce the PE wax is a copolymer, the total bio-based or renewable carbon of the PE may be contributed from renewable ethylene and/or renewable comonomers.

In one or more embodiments, the renewable source of carbon is one or more plant materials selected from the group consisting of sugar cane and sugar beet, maple, date palm, sugar palm, sorghum, American agave, corn, wheat, barley, sorghum, rice, potato, cassava, sweet potato, algae, fruit, materials comprising cellulose, wine, materials comprising hemicelluloses, materials comprising lignin, wood, straw, sugarcane bagasse, sugarcane leaves, corn stover, wood residues, paper, and combinations thereof.

In one or more embodiments, the renewable ethylene may be obtained by fermenting a renewable source of carbon to produce ethanol, which may be subsequently dehydrated to produce ethylene. Further, it is also understood that the fermenting produces, in addition to the ethanol, byproducts of higher alcohols. If the higher alcohol byproducts are present during the dehydration, then higher alkene impurities may be formed alongside the ethanol. Thus, in one or more embodiments, the ethanol may be purified prior to dehydration to remove the higher alcohol byproducts while in other embodiments, the ethylene may be purified to remove the higher alkene impurities after dehydration.

Biologically sourced ethanol, known as bio-ethanol, may be obtained by the fermentation of sugars derived from cultures such as that of sugar cane and beets, or from hydrolyzed starch, which is, in turn, associated with other cultures such as corn. It is also envisioned that the bio-based ethylene may be obtained from hydrolysis-based products of cellulose and hemi-cellulose, which can be found in many agricultural by-products, such as straw and sugar cane husks. This fermentation is carried out in the presence of varied microorganisms, the most important of such being the yeast Saccharomyces cerevisiae. The ethanol resulting therefrom may be converted into ethylene by means of a catalytic reaction at temperatures usually above 300° C. A large variety of catalysts can be used for this purpose, such as high specific surface area gamma-alumina. Other examples include the teachings described in U.S. Pat. Nos. 9,181,143 and 4,396,789, which are herein incorporated by reference in their entirety.

In one or more embodiments, the PE wax may exhibit a bio-based carbon content, as determined by ASTM D6866-18 Method B. of at least 5%. Further, other embodiments may include at least 10%, 20%, 40%, 50%, 60%, 80%, 90%, 95%, 99%, or 100% bio-based carbon. As discussed above, in embodiments where the PE wax comprises a copolymer, the total bio-based or renewable carbon of the PE wax may be contributed from renewable ethylene and/or renewable comonomers.

As discussed above, in one or more embodiments the structure-dependent properties of the wax (such as crystallinity, density, hardness, and melting point) may be dependent upon the identity of a starting material, such as a PE.

In one or more embodiments, the PE wax may have a melt flow rate, measured according to ASTM D1238 at 190° C. and 2.16 kg, of greater than 10,000 g/10 min. For instance, the PE wax may have a melt flow rate, measured according to ASTM D1238 at 190° C. and 2.16 kg, of a range having a lower limit selected from one of 10000, 15000, 20000, and 25000 g/10 min and an upper limit selected from one of 20000, 25000, 30000, 50000, and 75000 g/10 min, where any lower limit can be used with any mathematically-compatible upper limit.

In one or more embodiments, the PE wax may have a dropping point, measured according to ISO 2176, of 10 to 127° C. For instance, the PE wax may have a dropping point, measured according to ISO 2176, of a range having a lower limit selected from one of 100, 101, 102, 103, 104, 105, 106, 107, 108, 109, and 110° C. and an upper limit selected from one of 111, 112, 115, 116, 117, 118, 119, 120, 121, 122, 123, 124, 125, 126, and 127° C., where any lower limit can be used with any mathematically-compatible upper limit.

In one or more embodiments, the PE wax may have a solidification point (congealing point), measured according to ISO 2207, of 85 to 111° C. For instance, the PE wax may have a solidification point, measured according to ISO 2207, of a range having a lower limit selected from one of 85, 86, 87, 88, 89, and 90° C. and an upper limit selected from one of 89, 90, 92, 95, 98, 100, 101, 102, 103, 104, 105, 106, 107, 108, 109, 110, and 111° C., where any lower limit can be used with any mathematically-compatible upper limit.

In one or more embodiments, the PE wax may have a melting point, measured according to DIN 51007, of 80 to 110° C. For instance, the PE wax may have a solidification point, measured according to DIN 51007, of a range having a lower limit selected from one of 80, 85, 90, 95, 97, 99, 100, 101, 102, 103, and 104° C. and an upper limit selected from one of 103, 104, 105, 106, 107, 108, and 110° C., where any lower limit can be used with any mathematically-compatible upper limit.

In one or more embodiments, the PE wax may have a needle penetration, measured according to DIN 51579 or ASTM D1321 at 25° C., of 10 or less. For instance, the PE wax may have a needle penetration, measured according to DIN 51579 or ASTM D1321 at 25° C., of 10 or less, 8 or less, 7 or less, 6 or less, 5 or less, 4 or less, or 3 or less.

In one or more embodiments, the PE wax may have a dynamic viscosity, measured according to ISO 2555 at 140° C., of 5 to 2000 mPa-s. For instance, the PE wax may have a dynamic viscosity, measured according to ISO 2555 at 140° C. of a range having a lower limit selected from one of 5, 10, 20, 50, 100, 110, 115, 120, 125, 130, 135, 140, 175, 200, or 250 mPa-s and an upper limit selected from one of 130, 135, 140, 145, 150, 155, 160, 165, 180, 200, 250, 300, 500, 750, 1000, 1500, or 2000 mPa-s, where any lower limit can be used with any mathematically-compatible upper limit.

In one or more embodiments, the PE wax may have a density, measured according to ISO 1183-1 at 23° C., of 0.85 to 0.95 g/cm³. For instance, the PE wax may have a density, measured according to ISO 1183-1 at 23° C., of a range having a lower limit selected from one of 0.85, 0.86, 0.87, and 0.88 g/cm and an upper limit selected from one of 0.87, 0.88, 0.89, 0.90, 0.91, 0.92, 0.93, 0.94, and 0.95 g/cm³, where any lower limit can be used with any mathematically-compatible upper limit.

In one or more embodiments, the PE wax may have an acid value, measured according to ISO 2114, of 1.0 mg (KOH)/g or less. For instance, the PE wax may have an acid value, measured according to ISO 2114, of 1.0 mg (KOH)/g or less, 0.8 mg (KOH)/g or less, 0.6 mg (KOH)/g or less, or 0.4 mg (KOH)/g or less.

In one or more embodiments, the PE wax may have a saponification value, as measured according to ISO 3682, of 2.0 mg (KOH)/g or less. For instance, the PE wax may have a saponification value, measured according to ISO 3682, of 2.0 mg (KOH)/g or less, 1.8 mg (KOH)/g or less, 1.6 mg (KOH)/g or less, or 1.4 mg (KOH)/g or less.

In one or more embodiments, the PE wax may have a yellowness index, measured according to ISO 11664, of 30 or less. For instance, the PE wax may have a yellowness index, measured according to ISO 11664, of 30 or less, 28 or less, 25 or less, 22 or less, or 20 or less.

In one or more embodiments, the PE wax may have a flash point, measured with a Cleveland open cup apparatus according to ISO 2592, of 250° C. or more. For instance, the PE wax may have a flash point, measured with a Cleveland open cup apparatus according to ISO 2592, of 250° C. or more, 260° C. or more, 270° C. or more, or 280° C. or more.

In one or more embodiments, the PE wax may have a flash point, measured with a Pensky-Martens closed cup apparatus according to ISO 2719, of 200° C. or more. For instance, the PE wax may have a flash point, measured with a Pensky-Martens closed cup apparatus according to ISO 2719, of 200° C. or more, 210° C. or more, 220° C. or more, or 230° C. or more.

### Compositions

One or more embodiments in accordance with the present disclosure are directed to polymer compositions that contain one or more of the previously discussed PE waxes. In some embodiments, the polymer compositions may be used as hot melt adhesives, masterbatches, coatings, cosmetics, inks and paints, plastic additives, leather and textile finishes, and the like. In some embodiments the previously discussed PE waxes may be used as a replacement for one or more conventional waxes, such as synthetic PE waxes, Fischer-Tropsch waxes, paraffin waxes, and HDPE waxes, while either preserving or improving the performance of final product and application.

In one or more embodiments, the PE wax may exhibit a bio-based carbon content, as determined by ASTM D6866-18 Method B, of at least 5%. Further, other embodiments may include at least 10%, 20%, 40%, 50%, 60%, 80%, 90%, 95%, 99%, or 100% bio-based carbon. As discussed above, in embodiments where the PE wax comprises a copolymer, the total bio-based or renewable carbon of the PE wax may be contributed from renewable ethylene and/or renewable comonomers.

In one or more embodiments, a polymer composition may contain a PE wax in an amount of the range of 0.1 to 70% by weight (wt. %). For instance, the polymer composition may contain the PE wax in an amount of a range having a lower limit selected from one of 0.1, 0.2, 0.5, 1, 3, 4, 5, 10, 15, 20, 25, 30, 35, 40, and 45 wt. % and an upper limit selected from one of 10, 12, 15, 20, 25, 30, 35, 40, 45, 50, 55, 60, 65, and 70 wt. %, where any lower limit can be used with any mathematically-compatible upper limit.

In one or more embodiments, the PE wax may optionally be oxidized. Such oxidation (or functionalization) may result in an increased water solubility for the wax or ability to be emulsified.

In those embodiments, the PE wax may have an acid value, measured according to ISO 2114, of 15.0 mg (KOH)/g or more. For instance, the PE wax may have an acid value, measured according to ISO 2114, of 15 mg (KOH)/g or more, 20 mg (KOH)/g or more, 50 mg (KOH)/g or more, or 80 mg (KOH)/g or less.

In those embodiments, the PE wax may have a saponification value, as measured according to ISO 3682, of 15 mg (KOH)/g or less. For instance, the PE wax may have a saponification value, measured according to ISO 3682, of 15 mg (KOH)/g or less, 50 mg (KOH)/g or less, 80 mg (KOH)/g or less, or 100 mg (KOH)/g or less.

In one or more embodiments, a polymer composition may exhibit a biobased carbon content, as determined by ASTM D6866-18 Method B, of at least 5%. Further, other embodiments may include at least 10%, 20%, 40%, 50%, 60%, 80%, 90%, 95%, 99%, or 100% bio-based carbon. The biobased carbon content may be provided by the PE wax and/or other components in the polymer composition.

With the benefit of this disclosure, a person of ordinary skill in the art will appreciate that the formulation of a polymer composition is dependent upon the ultimate application of said composition.

### Hot Melt Adhesives and Coatings

Polymer compositions of one or more embodiments may be hot melt adhesive compositions. In one or more embodiments, the hot melt adhesive compositions may be formulated with one or more polymers, one or more tackifiers, one or more green PE waxes (as described previously), and, optionally, one or more additional performance modifiers. The selection of the components of a hot melt adhesive composition is partially determined by the particular application of the composition.

In one or more embodiments, a hot melt adhesive composition may contain one of the polymers in an amount that ranges from a lower limit of any of 5, 10, 15, 20, 25, 30, and 35 wt. %, to an upper limit of any of 30, 35, 40, 45, 50, 55, 60, and 65 wt. %, where any lower limit may be paired with any upper limit. In particular embodiments, the polymer may be present in the holt melt adhesive composition in an amount of the range of 25 to 45 wt %.

In one or more embodiments, hot melt adhesive compositions may comprise one or more polymers selected from the group consisting of ethyl vinyl acetate (EVA) copolymers, ethylene copolymers (such as ethylene-acrylic ester copolymers, including ethylene-butyl acrylate copolymers (EBA) and ethylene-methyl acrylate copolymers (EMA)), polyolefins (such as poly α-olefins, including PE and polypropylene (PP), among others), styrene block copolymers (such as styrene-isoprene-styrene (SIS) and styrene-butadiene-styrene (SBS)). In particular embodiments, hot melt adhesive compositions may comprise an EVA and a polyolefin.

The EVA copolymers of the hot melt adhesive compositions of one or more embodiments may incorporate various ratios of ethylene and vinyl acetate, and may, in some embodiments, include one or more additional comonomers. In one or more embodiments, the EVA copolymer may be derived from fossil sources. In some embodiments, the EVA copolymer may be partially, or completely derived from renewable sources.

Hot melt adhesive compositions in accordance with one or more embodiments of the present disclosure may include biobased EVA copolymers incorporating various ratios of ethylene and vinyl acetate. In one or more embodiments, hot melt adhesive compositions in accordance with one or more embodiments may include a biobased EVA copolymer, wherein the percent by weight (wt. %) of ethylene in the biobased EVA ranges from a lower limit selected from any one of 30 wt. %, 40 wt. %, 45 wt. %, 50 wt. %, 55 wt. %, 60 wt. %, 66 wt. %, 68 wt. %, and 72 wt. %, to an upper limit selected from any one of 68 wt. %, 70 wt. %, 72 wt. %, 75 wt. %, 78 wt. %, 80 wt. %, 82 wt. %, 85 wt. %, 88 wt. %, and 92 wt. %, where any lower limit may be paired with any mathematically-compatible upper limit. Similarly, hot melt adhesive compositions in accordance with one or more embodiments of the present disclosure may include a biobased EVA copolymer having a wt. % of vinyl acetate content as determined by ASTM D5594 that ranges from a lower limit selected from any one of 8 wt. %, 12 wt. %, 15 wt. %, 18 wt. %, 20 wt. %, 22 wt. %, 25 wt. %, 28 wt. %, 30 wt. % and 32 wt. % to an upper limit selected from any one of 28 wt. %, 30 wt. %, 33 wt. %, 35 wt. %, 40 wt. %, 45 wt. %, and 50 wt. %, where any lower limit may be paired with any mathematically-compatible upper limit. In some embodiments, biobased EVA may be selected from commercially available resins by Braskem such as SVT2180 or SVT2145R.

EVA copolymers of the hot melt adhesive compositions of one or more embodiments may have a biobased carbon content, as determined by ASTM D6866-18 Method B, that ranges from a lower limit selected from any one of 5%, 10%, 20%, 40%, 50%, and 55%, to an upper limit selected from any one of 50%, 55%, 60%, 80%, 95%, 99%, and 100% where any lower limit may be paired with any upper limit. The total biobased or renewable carbon in the EVA polymer may be contributed from a biobased ethylene and/or a biobased vinyl acetate. It is understood that if at least a portion of the ethylene and/or the vinyl acetate is derived from a renewable source, it can be considered a biobased EVA, even if a fossil based ethylene and/or vinyl acetate is present in the polymerization process. Further, while particular embodiments of the present disclosure may be directed to use of biobased EVA copolymers in the production of hot melt adhesive compositions, it is also understood that one or more other components may also be formed from renewable sources or one or more other components may be formed from fossil sources. The total biobased carbon content of the final composition and article, discussed below, may thus be based on consideration of all components.

Biobased ethylene may be sourced and/or produced as discussed previously in this disclosure. Similar, or the same, sources of renewable carbon may be used to produce biobased vinyl acetate included in the biobased EVA copolymers of one or more embodiments.

Biobased vinyl acetate may be produced by producing acetic acid by oxidation of ethanol (which may be formed as described above) followed by reaction of ethylene and acetic acid to acyloxylate the ethylene and arrive at vinyl acetate. Further, it is understood that the ethylene reacted with the acetic acid may also be formed from a renewable source as described above. Additional details about oxidation of ethanol to form acetic acid may be found in U.S. Pat. No. 5,840,971 and Selective catalytic oxidation of ethanol to acetic acid on dispersed Mo—V—Nb mixed oxides. Li X, Iglesia E. Chemistry; 2007; 13(33):9324-30.

Vinyl acetate in accordance with the present disclosure may also be generated by the esterification of acetic acid obtained from a number of natural sources, including conversion of fatty acid, as described in The Production of Vinyl Acetate Monomer as a Co-Product from the Non-Catalytic Cracking of Soybean Oil, Benjamin Jones, Michael Linnen, Brian Tande and Wayne Seames, Processes, 2015, 3, 61-9-633. Further, the production of acetic acid from fermentation performed by acetogenic bacteria, as described in Acetic acid bacteria: A group of bacteria with versatile biotechnological applications, Saichana N, Matsushita K, Adachi O, Frébort I, Frebortova J.; Biotechnol Adv. 2015 Nov. 1; 33(6 Pt 2):1260-71 and Biotechnological applications of acetic acid bacteria. Raspor P. Goranovic D. Crit Rev Biotechnol.; 2008; 28(2):101-24.

Biobased EVA copolymers in accordance with the present disclosure may have a melt index (I₂) as determined by ASTM D1238 with a load of 2.16 kg at 190° C. that may range of a lower limit selected from any one of 1.5 g/10 min, 2.0 g/10 min and 3.0 g/10 min, to an upper limit selected from any one of 5 g/10 min, 10 g/10 min, 20 g/10 min, 25 g/10 min, 40 g/10 min, 50 g/10 min, 100 g/10 min, 200 g/10 min, 400 g/10 min, 500 g/10 min, and 900 g/10 min, where any lower limit can be used with any upper limit. In particular embodiments, a biobased EVA copolymer may have a vinyl acetate content as determined by ASTM D5594 of 16 wt % to 45 wt %; and a melt index (I₂) as determined by ASTM D1238 in the range of 1.5 g/10 min to 5 g/10 min measured with a load of 2.16 kg at 190° C.

Biobased EVA copolymers, in accordance with the present disclosure may have a density as determined by ASTM D792 that may range of a lower limit selected from any one of 0.9 g/cm³, 0.91 g/cm³, 0.92 g/cm³, and 0.93 g/cm³ to an upper limit selected from any one of 0.94 g/cm³, 0.95 g/cm³, 0.96 g/cm³, or 0.97 g/cm³, where any lower limit can be used with any upper limit.

In one or more embodiments, hot melt adhesive compositions may contain a mixture of biobased EVA and “fossil EVA” copolymers derived from traditional fossil fuel sources or otherwise differentiated from the biobased EVA described above. In some embodiments, an adhesive composition may contain a fossil EVA copolymer a percent by weight (wt. %) of the composition that ranges from a lower limit of 1 wt. %, 5 wt. %, 8 wt. %, 10 wt. %, 15 wt. %, 20 wt. %, 25 wt. %, or 30 wt. %, to an upper limit of 10 wt. %, 15 wt. %, 20 wt %, 25 wt. %, 30 wt. %, 35 wt. %, 40 wt. %, or 50 wt. %, where any lower limit may be paired with any upper limit. In particular embodiments, hot melt adhesive compositions in accordance with the present disclosure may include 1 to 40 wt. % of fossil EVA.

In particular embodiments, fossil EVA copolymers in accordance with the present disclosure may include an amount of vinyl acetate as determined by ASTM D5594 that ranges from a lower limit selected from any one of 12 wt. %, 16 wt. %, 17 wt. %, 20 wt. %, 26 wt. %, 28 wt. %, and 35 wt. %, to an upper limit selected from any one of 30 wt. %, 35 wt. %, 40 wt. %, and 45 wt. %, where any lower limit may be paired with any upper limit.

In particular embodiments, adhesive compositions may include a fossil EVA that exhibits a vinyl acetate content as determined by ASTM D5594 of 16 to 45 wt. %, and a melt index (I₂) as determined by ASTM D1238 in the range of 2.5 to 900 g/10 min measured with a load of 2.16 kg at 190° C. In other embodiments, fossil EVA copolymers in accordance with the present disclosure may have a melt index (I₂) as determined by ASTM D1238 as measured with a load of 2.16 kg at 190° C. that ranges from 150 g/10 min to 800 g/10 min. In some embodiments, fossil EVA resins may be selected from commercially available resins by Braskem such as HM728, 3019PE, 8019PE, PN2021, HM150, HM728F, and HM2528.

Fossil EVA copolymers, in accordance with the present disclosure may have a density as determined by ASTM D1505/D792 that may range of a lower limit selected from any one of 0.91 g/cm³, 0.915 g/cm³ and 0.92 g/cm³ to an upper limit selected from any one of 0.95 g/cm³, 0.96 g/cm³, or 0.97 g/cm³, where any lower limit can be used with any upper limit.

In one or more embodiments, hot melt adhesive compositions may further comprise a secondary polymer selected from the group consisting of ethyl vinyl acetate (EVA) copolymers, ethylene copolymers (such as ethylene-acrylic ester copolymers, including ethylene-butyl acrylate copolymers (EBA) and ethylene-methyl acrylate copolymers (EMA)), polyolefins (such as poly α-olefins, including PE and polypropylene (PP), among others), and styrene block copolymers (such as styrene-isoprene-styrene (SIS) and styrene-butadiene-styrene (SBS)). In particular embodiments, the secondary polymer may be LLDPE or metallocene PE.

In one or more embodiments, a hot melt adhesive composition may contain the second polymer in an amount that ranges from a lower limit of any of 0.1, 0.5, 1, 2, 3, 5, 7, and 10 wt. %, to an upper limit of any of 5, 6, 7, 8, 9, 10, 15, 25, and 50 wt. %, where any lower limit may be paired with any upper limit. In particular embodiments, the polymer may be present in the holt melt adhesive composition in an amount of the range of 0.1 to 10 wt %.

Secondary polymers in accordance with one or more embodiments of the present disclosure may have a melt index (I₂), in accordance with ASTM D1238 as measured with a load of 2.16 kg at 190° C., that ranges from a lower limit selected from any one of 2 g/10 min, 2.5 g/10 min, 25 g/10 min, 100 g/10 min, 150 g/10 min, and 200 g/10 min, to an upper limit selected from 250 g/10 min, 300 g/10 min, 400 g/10 min, 500 g/10 min, and 900 g/10 min, where any lower limit may be paired with any upper limit.

In one or more embodiments, hot melt adhesive compositions may comprise one or more tackifiers. Tackifiers in accordance with the present disclosure may be a chemical compound or low molecular weight polymer that enhances the adhesion of a hot melt adhesive composition. Tackifiers include any compatible resins or mixtures thereof such as natural and modified rosins including gum rosin, wood rosin, tall oil rosin, distilled rosin, hydrogenated rosin, dimerized rosin, rosin esters, and polymerized rosin; glycerol and pentaerythritol esters of natural and modified rosins, including phenolic-modified rosins and rosin esters; monomeric resins; polymers and copolymers of natural terpenes such as pinene; terpene resins; hydrogenated polyterpene resins; phenolic modified terpene resins and hydrogenated derivatives thereof; indene-coumarone resins; aliphatic petroleum hydrocarbon resins; hydrogenated aliphatic petroleum hydrocarbon resins; C5/C9 hydrocarbon resins, including cyclic or acylic C5 resins and aromatic modified acyclic or cyclic resins, cyclic petroleum hydrocarbon resins and the hydrogenated derivatives, and the like. In some embodiments, tackfiers may be selected from hydrocarbon resins. In other embodiments tackfiers may be selected from commercially available hydrocarbon resins by Braskem such as resins from the UNILENE® family, including Unilene A80, Unilene A90, Unilene A100 or Unilene A120.

In some embodiments, a hot melt adhesive composition may contain a tackifier in an amount of a range having a lower limit of any of 1, 2, 3, 5, 7, 10, 12, 15, 18, 20, 22, 25, 30, 35, and 40 wt. % and an upper limit of any of 5, 7, 10, 15, 20, 25, 30, 35, 40, 45, 50, 55, 60, 65, and 70 wt. %, where any lower limit may be paired with any mathematically-compatible upper limit.

In one or more embodiments, tackifiers may be formulated as a concentrate, or “tackifier masterbatch” that is combined with other polymers and/or additives to prepare a hot melt composition. Tackifier masterbatches may be prepared by any conventional process of mixing resins, such as solubilization and extrusion processes. In one or more embodiments, tackifier masterbatches may be formulated with a tackifier and any suitable polymer, as discussed previously, which has good compatibility with the other components of the hot melt adhesive composition. In particular embodiments the polymer may be an EVA copolymer. Tackfier masterbatches in accordance with one or more embodiments of the present disclosure may contain tackifiers at a percent by weight (wt. %) of the masterbatch that ranges from 30 wt. % to 70 wt. % and a base polymer at a percent by weight (wt. %) of the masterbatch that ranges from 30 wt. % to 70 wt. %. In one or more embodiments, a hot melt adhesive composition may comprise a tackifier masterbatch in an amount of the range of 20 wt. % to 70 wt. %.

Hot melt adhesive compositions in accordance with one or more embodiments of the present disclosure may include one or more additional performance modifiers, such as a secondary wax (in addition to the previously described green PE wax), antioxidants, stabilizer, and other additives.

Hot melt adhesive compositions of one or more embodiments may optionally incorporate one or more secondary waxes. Secondary waxes suitable for use in the present invention include paraffin waxes, microcrystalline waxes, high density low molecular weight polyethylene waxes, by-product polyethylene waxes, Fischer-Tropsch waxes, oxidized Fischer-Tropsch waxes, and functionalized waxes such as hydroxystearamide waxes and fatty amide waxes. It is common in the art to use the terminology synthetic high melting point waxes to include high density low molecular weight polyethylene waxes, by-product polyethylene waxes and Fischer-Tropsch waxes. Modified waxes, such as vinyl acetate modified and maleic anhydride modified waxes may also be used. Example waxes useful in the practice of the present embodiments will have a melting point of from about 50 to 600° C., and may have an oil content of less that about 0.5.

In one or more embodiments, hot melt adhesive compositions in accordance with one or more embodiments may contain a secondary wax in an amount of a range having a lower limit of any of 0.1, 1, 2, 3, 4, 5, 7, 10, and 15 wt. % and an upper limit of any of 10, 12, 15, 18, 20, and 25 wt. %, where any lower limit can be used with any mathematically-compatible upper limit.

Hot melt adhesive compositions in accordance with one or more embodiments of the present disclosure may include fillers and additives that modify various physical and chemical properties when added to the polymer composition during blending that include one or more polymer additives such as processing aids, lubricants, antistatic agents, clarifying agents, nucleating agents, beta-nucleating agents, slipping agents, antioxidants, compatibilizers, antacids, light stabilizers such as HALS, IR absorbers, whitening agents, inorganic fillers, organic and/or inorganic dyes, anti-blocking agents, processing aids, flame-retardants, plasticizers, biocides, adhesion-promoting agents, metal oxides, mineral fillers, glidants, oils, antioxidants, antiozonants, accelerators, and vulcanizing agents.

Hot melt adhesive compositions in accordance with one or more embodiments of the present disclosure may include one or more inorganic fillers such as talc, glass fibers, marble dust, cement dust, clay, carbon black, feldspar, silica or glass, fumed silica, silicates, calcium silicate, silicic acid powder, glass microspheres, mica, metal oxide particles and nanoparticles such as magnesium oxide, antimony oxide, zinc oxide, inorganic salt particles and nanoparticles such as barium sulfate, wollastonite, alumina, aluminum silicate, titanium oxides, calcium carbonate, polyhedral oligomeric silsesquioxane (POSS), or recycled EVA. As defined herein, recycled EVA may be derived from regrind materials that have undergone at least one processing method such as molding or extrusion and the subsequent sprue, runners, flash, rejected parts, and the like, are ground or chopped.

Hot melt adhesive compositions in accordance with one or more embodiments of the present disclosure may particularly include one or more of a stabilizer and an antioxidant. In some embodiments, the hot melt adhesive composition may include the stabilizer and/or the antioxidant in an amount of a range having a lower limit of any of 0.01, 0.1, 0.2, 0.5, 0.8, 1.0, and 1.2 wt % and an upper limit of any of 0, 1.0, 1.2, 1.4, 1.6, 1.8, 2.0, and 2.5 wt. %, where any lower limit can be used with any mathematically-compatible upper limit.

In one or more embodiments, polymer compositions in accordance with the present disclosure may contain a percent by weight of the total composition (wt. %) of one or more fillers that ranges from a lower limit selected from one of 0.02 wt. %, 0.05 wt. %, 1.0 wt. %, 5.0 wt. %, 10.0 wt. %, 15.0 wt. %, and 20.0 wt. %, to an upper limit selected from one of 25.0 wt. %, 30.0 wt. %, 40.0 wt. %, 50.0 wt. %, 60.0 wt. %, and 70.0 wt. %, where any lower limit can be used with any upper limit.

Hot melt adhesive compositions in accordance with the present disclosure may be prepared in any conventional mixture device. In one or more embodiments, hot melt adhesive composition may be prepared by mixture in conventional Sigma mixers, horizontal mixers, kneaders, banbury mixers, mixing rollers, extruders, and the like.

In one or more embodiments, all the components may be mixed together in a single step. In other embodiments, when a secondary polymer is present in the composition, there can be a pre-mixture step of a first polymer and the secondary polymer in a conventional mixture device, such as in extruders, alternatively being pelletized, prior to a mixture with other components in a subsequent mixture step.

The hot melt adhesive compositions of one or more embodiments may be prepared in any known process for adhesive formulation such as compounding with Sigma mixers, horizontal mixers, kneaders, blenders, extruders, and any other available manufacturing processes.

In one or more embodiments, the hot melt adhesive composition may have an open time, measured according to ASTM D4497, of 1 to 25 s. For instance, the hot melt adhesive composition may have an open time, measured according to ASTM D4497, of a range having a lower limit selected from one of 1, 2, 3, 5, 8, 10, 12 and 15 s and an upper limit selected from one of 10, 12, 15, 18, 20, 22, and 25 s, where any lower limit can be used with any mathematically-compatible upper limit.

In one or more embodiments, the hot melt adhesive composition may have a shear adhesion failure temperature of 30 to 120° C. For instance, the hot melt adhesive composition may have a shear adhesion failure temperature of a range having a lower limit selected from one of 30, 40, 50, 60, 70, 80, 85, 90, and 95° C. and an upper limit selected from one of 90, 95, 100, 105, 110, 115, and 120° C., where any lower limit can be used with any mathematically-compatible upper limit.

In one or more embodiments, the hot melt adhesive composition may have a softening point, measured according to ASTM D6493, of 80 to 130° C. For instance, the hot melt adhesive composition may have a softening point, measured according to ASTM D6493, of a range having a lower limit selected from one of 80, 85, 90, 95, 100, 105, and 110° C. and an upper limit selected from one of 110, 115, 120, 125, and 130° C., where any lower limit can be used with any mathematically-compatible upper limit.

In one or more embodiments, the hot melt adhesive composition may have a viscosity, measured according to ASTM 3236 at 150° C., of 1500 to 5000 cPs. For instance, the hot melt adhesive composition may have a viscosity, measured according to ASTM 3236 at 150° C. of a range having a lower limit selected from one of 1500, 1750, 2000, 2250, 2500, 2750, and 3000 cPs and an upper limit selected from one of 2500, 3000, 3500, 4000, 4500, and 5000, where any lower limit can be used with any mathematically-compatible upper limit.

The hot melt adhesives of one or more embodiments may be used to generate articles, such as multilayer structures, by bonding similar or dissimilar substrates, which may include applying a hot melt adhesive composition to at least one substrate and bonding the layers together. For example, the adhesive composition may be melted and applied to the at least one substrate to which it is being bound. Substrates may take the form of films, blocks, sheets, fiber, thread, strip, ribbon, coating, foil, band, and the like. While there are no practical limits on the type of substrate that may be bonded using adhesive compositions in accordance with the present disclosure, exemplary substrates may include fabrics, non-woven materials, polymers and polymeric materials such as polyurethane, EVA, polypropylene, polyethylene, polyvinylchloride, polyester, polyamide, polyolefin, polyacrylic, polyester, polyvinyl chloride, polystyrene, cellulosics such as wood, metal, cardboard, paper, kraft and the like.

### Filler Masterbatch

Polymer compositions of one or more embodiments may be a filler masterbatch (also known as a CaCO₃ filler masterbatch). In one or more embodiments, the filler masterbatch may be suitable for use in the plastics industry as being a suitable replacement for a portion of virgin polymer that would otherwise be needed and/or to provide desired properties to the end product. In one or more embodiments, the hot melt adhesive compositions may be formulated with calcium carbonate, one or more green PE waxes (as described previously), one or more lubricants, one or more coupling agents, and, optionally, one or more additives. The selection of the components of a filler masterbatch composition is partially determined by the particular application of the composition. In some embodiments, the filler masterbatch may be similar to known masterbatch formulations but with the replacement of a fossil wax with the green PE wax, without affecting dispersing and manufacturing process. The filler masterbatch may be used, for example, directly into injection molding processes or mixed with other resins during a molding process.

In one or more embodiments, a filler masterbatch may contain calcium carbonate (lime carbonate) in an amount that ranges from a lower limit of any of 40, 45, 50, 55, 60, 65, and 70 wt. %, to an upper limit of any of 60, 65, 70, 75, 80, and 85 wt. %, where any lower limit may be paired with any mathematically-compatible upper limit. In particular embodiments, the calcium carbonate may be present in the filler masterbatch in an amount of the range of 50 to 85 wt %.

As discussed above, in one or more embodiments, a filler masterbach may contain a PE wax in an amount of the range of 0.1 to 20% by weight (wt. %). In particular embodiments, the filler masterbatch may contain the PE wax in an amount of a range having a lower limit selected from one of 0.1, 1, 2, 3, 5, 7, and 10 wt. % and an upper limit selected from one of 5, 7, 10, 12, 15, 18, and 20 wt. %, where any lower limit can be used with any mathematically-compatible upper limit.

In one or more embodiments, a filler masterbatch may contain one or more coupling agents. The coupling agents are not particularly limited and may be any such agents known to a person of ordinary skill in the art, for instance silanes, aluminates or titanates. In one or more embodiments, a filler masterbatch may contain a coupling agent in an amount that ranges from a lower limit of any of 1, 2, or 3 wt. %, to an upper limit of any of 5, 8, or 10 wt. %, where any lower limit may be paired with any mathematically-compatible upper limit.

In one or more embodiments, a filler masterbatch may contain one or more oxidation inhibitors. The oxidation inhibitors are not particularly limited and may be any such inhibitors known to a person of ordinary skill in the art, for instance hindered phenols, triaryl phosphites, aromatic amines, and hydroxylamines. In one or more embodiments, a filler masterbatch may contain an oxidation inhibitor in an amount that ranges from a lower limit of any of 1, 3, or 5 wt. %, to an upper limit of any of 8, 10, 12, and 15 wt % where any lower limit may be paired with any mathematically-compatible upper limit.

In one or more embodiments, a filler masterbatch may further comprise a polyolefin. The polyolefin of one or more embodiments is not particularly limited and may be a homopolymer or copolymer of poly α-olefins, including PE and PP.

In one or more embodiments, a filler masterbatch may contain the polymer in an amount that ranges from a lower limit of any of 10, 12, 15, 18, and 20 wt. %, to an upper limit of any of 20, 22, 25, 28, and 30 wt. %, where any lower limit may be paired with any mathematically-compatible upper limit.

Filler masterbatches in accordance with one or more embodiments of the present disclosure may include fillers and additives that modify various physical and chemical properties when added to the polymer composition during blending that include one or more polymer additives such as processing aids, lubricants, antistatic agents, clarifying agents, nucleating agents, beta-nucleating agents, slipping agents, compatibilizers, antacids, light stabilizers such as HALS, IR absorbers, whitening agents, inorganic fillers, organic and/or inorganic dyes, anti-blocking agents, processing aids, flame-retardants, plasticizers, biocides, adhesion-promoting agents, metal oxides, mineral fillers, glidants, oils, antioxidants, antiozonants, accelerators, blowing agents, and vulcanizing agents. Generally, one or more of such additives may be included at an amount ranging from 0.1 to 30 wt %. In one or more embodiments, a filler masterbatch may include an antioxidant present in an amount ranging from 1 to 10 wt %, a blowing agent present in an amount ranging from 1 to 30 wt %, and a UV stabilizer present in an amount ranging from 1 to 20 wt %.

Further, while one or more embodiments are directed to filler masterbatch compositions, it is also envisioned that the PE wax described herein may also be used in other masterbatch compositions not limited to filler masterbatches. Masterbatch compositions including the described PE wax may have broad application with any of polyvinyl chloride, polyethylene, polypropylene, polyamides, polyoxymethylene, polystyrene, acrylonitrile butadiene styrene, polyethylene terephthalate, ethylene vinyl acetate, polyisobutylene, styrene butadiene, and polycarbonate. In particular, the presently described waxes may be used to disperse pigments or other additives, in particular, by improving the pigment wettability and its distribution. The wax also reduces the viscosity of the masterbatch melt, resulting in better pigment distribution and increased color efficiency. By reducing the viscosity caused by the addition of PE wax, the potential for increasing pigment content in the masterbatch produced without any negative effect may be provided. In addition, with the addition of wax, the viscosity of the masterbatch melt containing polyethylene wax may be reduced, resulting in easier mixing of the device and a significant increase in production rate. As a result of the capillary effect, the wax enters the intercostal space, so a lower viscosity wax is more advantageous than a higher viscosity wax. Thus, addition of PE wax in masterbatch may provide following advantages: increased color intensity, excellent dilution capability, great movie transparency, reduce stain formation, reduce clutter, reduction of clump reorganization. in downstream processes, and easy shipping.

### Cosmetics

Polymer compositions of one or more embodiments may be cosmetic compositions, such as solid lip treatment or balm, lipsticks, and other lip or skin products. In one or more embodiments, a cosmetic composition may be formulated with one or more green PE waxes (as described herein previously), one or more skin condition agents or emollients, one or more liquid components, optionally one or more surfactants, optionally one or more colorants,

In one or more embodiments, the amount of green PE wax present in the cosmetic composition may depend on the type of cosmetic composition, and may broadly range from 3 to 20 wt %, such as from a lower limit of any of 3, 4, 5, or 8 wt %, to an upper limit of any of 12, 15, or 20 wt %, where any lower limit can be used in combination with any upper limit.

Selection of the liquid component may depend on the type of cosmetic, and may include one or more oils, glycols or glycol esters (such as propylene glycol or neopentyl glycol diheptanoate), water, and/or water-soluble solvent (such as alcohols). Similarly, the amount of liquid component may depend on the type of cosmetic composition and may broadly range from 1 to 80 wt %, with a lower limit of any of 1, 5, 10, 15, 20, 30, or 40 wt % and an upper limit of any of 20, 30, 35, 40, 60, 70, or 80 wt %, where any lower limit can be used in combination with any upper limit.

In one or more embodiments, the skin-conditioning agent and/or emollient may include, for example, hydrogenated polyisobutene, stearates such as isopropyl hydroxystearate, silicones and silicone elastomers. The amount of skin-conditioning agent and/or emollient used may depend on the type of cosmetic composition, and may generally range from 2 to 90 wt %, such as from a lower limit of any of 2, 5, 10, 20, 25, or 30 wt % to an upper limit of any of 20, 30, 40, 50, 60, 70, 80, or 90 wt %, where any lower limit can be used in combination with any upper limit.

Optionally, the cosmetic compositions may include various other components such as surfactants or colorants, depending on the types of cosmetic composition. For example, a surfactant may optionally be present in an amount up to 8 wt %, such as from a lower limit of any of 0.5, 1, 2, or 4 wt % to an upper limit of any of 4, 6, or 8 wt %, where any lower limit can be used in combination with any upper limit. A colorant may be present in an amount up to 30 wt %, such as from a lower limit of any of 1, 2, 5, or 10 wt % to an upper limit of any of 10, 15, 20, 25, or 30 wt %, where any lower limit can be used in combination with any upper limit.

In one or more embodiments, the cosmetic composition is a solid lip cosmetic that is formulated with a green PE wax in an amount ranging from 3 to 12 wt %, hydrogenated polyisobutene in an amount ranging from 25 to 50 wt %, glycol in an amount ranging from 1 to 20 wt %, silicone in an amount ranging from 20 to 70 wt %, and a surfactant in an amount at up to 8 wt %.

In one or more embodiments, the cosmetic composition is a solid lip cosmetic that is formulated with a green PE wax in an amount ranging from 5 to 12 wt %, hydrogenated polyisobutene in an amount ranging from 10 to 30 wt %, silicone in an amount ranging from 30 to 70 wt %, and a surfactant in an amount ranging from 0.5 to 8 wt %.

In one or more embodiments, the cosmetic composition is a lipstick that is formulated with a green PE wax in an amount ranging from 4 to 20 wt %, oils in an amount ranging from 30 to 80 wt %, colorants in an amount up to 30 wt %, stearates in an amount up to 30 wt %, and isopropyl hydroxystearate in an amount ranging from 2 to 20 wt %.

In one or more embodiments, the cosmetic composition is a lip or skin cream that is formulated with green PE wax in an amount ranging from 5 to 20 wt %, volatile oil in an amount ranging from 20 to 35 wt %, and silicone elastomer in an amount ranging from 5 to 30 wt %.

In one or more embodiments, the cosmetic composition is a lip or skin cream that is formulated with green PE wax in an amount that is at least 20 wt %, volatile oil in an amount up to 70 wt %, and water and/or a water soluble solvent in an amount up to 20 wt %.

### Inks

Polymer compositions of one or more embodiments may be solvent-based or solid paints or inks. In conventional printing, the resistance to abrasion and scratches of an ink may be improved by the inclusion of a wax, such as synthetic PE wax. However, the use of non-aromatic solvents, which are generally preferred due to the health hazards associated with aromatic solvents, negatively impacts the stability of said synthetic PE waxes. In contrast, the inks of one or more embodiments may replace the known synthetic PE waxes with one or more green PE waxes (as discussed previously). The use of the green PE waxes may not only improve the recycled content of the final ink formulation and provide more sustainable ink production, but also yield inks that have excellent storage stability.

The selection of the components of the ink composition may be determined by the particular application of the composition. In some embodiments, the ink compositions may be similar to known ink compositions but with the replacement of a fossil wax for the green PE wax. Offset ink compositions of one or more embodiments may include one or more pigments, one or more green PE waxes, one or more secondary waxes, a binder, and ink oil. When a matte ink is desired, a matte vehicle may be used in place of the one or more secondary waxes.

In one or more embodiments, an ink composition may contain one or more pigments. The pigments are not particularly limited and may be any such pigments known to a person of ordinary skill in the art, for instance azo pigments such as condensed and chelate azo pigments; polycyclic pigments such as phthalocyanines, anthraquinones, quinacridones, thioindigoids, isoindolinones, and quinophthalones; nitro pigments; daylight fluorescent pigments; carbonates; chromates; titanium oxides; zinc oxides; iron oxides and carbon black. In embodiments, the pigment may include carbon black and pigments capable of generating a cyan, magenta and yellow ink. Suitable commercially available pigments include, for example, Pigment Red 81, Pigment Red 122, Pigment Yellow 13, Pigment Yellow 14, Pigment Yellow 17, Pigment Yellow 74, Pigment Yellow 83, Pigment Yellow 128, Pigment Yellow 138, Pigment Orange 5, Pigment Orange 30, Pigment Orange 34, Pigment Blue 15:4 and Pigment Blue 15:3. In one or more embodiments, an ink composition may contain one or more pigments in an amount that ranges from a lower limit of any of 5, 10, 20, 30, and 40 wt. % to an upper limit of any of 30, 40, 50, 60 wt. %, where any lower limit may be paired with any mathematically-compatible upper limit.

As discussed above, in one or more embodiments, an ink composition may contain a PE wax in an amount of the range of 0.1 to 10% by weight (wt. %). In particular embodiments, the ink composition may contain the PE wax in an amount of a range having a lower limit selected from one of 0.1, 1, 1.5, 2, or 3 wt. % and an upper limit selected from one of 2, 2.5, 3, 5, or 10 wt. %, where any lower limit can be used with any mathematically-compatible upper limit.

In one or more embodiments, an ink composition may contain a secondary wax, which is not particularly limited. In some embodiments, the secondary wax may be a synthetic PE wax or a PTFE wax. The ink composition of one or more embodiments may include the secondary wax in an amount of the range of 0.1 to 3% by weight (wt. %). In particular embodiments, the ink composition may contain the secondary wax in an amount of a range having a lower limit selected from one of 0.1, 0.5, 1, and 1.5 wt. % and an upper limit selected from one of 1, 1.5, 2, 2.5, and 3 wt. %, where any lower limit can be used with any mathematically-compatible upper limit.

In one or more embodiments, an ink composition may contain one or more binders. The binders are not particularly limited and may be any such binders known to a person of ordinary skill in the art, for instance varnishes that may include one or more solvents and one or more resins such as acrylic resins, vinyl resins, or hydrocarbon-soluble resins. In one or more embodiments, an ink composition may contain a binder in an amount of a range having a lower limit selected from one of 20, 30, or 50 wt. % and an upper limit selected from one of 50, 70, or 80 wt. %, where any lower limit can be used with any mathematically-compatible upper limit.

In one or more embodiments, an ink composition may contain one or more ink oils. The ink oils are not particularly limited and may be any such ink oils known to a person of ordinary skill in the art, for instance vegetable oils, mineral oils, etc. In one or more embodiments, an ink composition may contain an ink oil in an amount of a range having a lower limit selected from one of 2, 4, and 5 wt. % and an upper limit selected from one of 5, 7, 8, or 10 wt. %, where any lower limit can be used with any mathematically-compatible upper limit.

In one or more embodiments, an ink composition may optionally contain a matte vehicle. The matte vehicle is not particularly limited and may be any such matte vehicle known to a person of ordinary skill in the art, for instance fumed silica. In one or more embodiments, an ink composition may contain a matte vehicle in an amount of a range having a lower limit selected from one of 1, 3, and 5 wt. % and an upper limit selected from one of 5, 8, 10, or 15 wt. %, where any lower limit can be used with any mathematically-compatible upper limit.

Ink compositions in accordance with one or more embodiments of the present disclosure may include fillers and additives that modify various physical and chemical properties when added to the ink composition. The ink compositions of the present disclosure may possess an improved scratch resistance relative to a comparable composition formulated without the PE wax of the present disclosure. In one or more embodiments, the ink compositions may pass at least 5, 15, or 50 passes on an ink surface interaction tester, i.e., an applied ink does not rub off during such passes through the tester.

### PVC Applications and Processing

Polymer compositions of one or more embodiments may be compositions for PVC processing. In the PVC processing compositions of one or more embodiments, green PE waxes (such as those described previously) may be included as an alternative, or in addition, to conventional lubricants. Compared with fatly acid lubricants, green PE wax may provide no adverse effects on melt tension and Vicat softening point, and provides excellent anti-adhesion and flow control.

In some embodiments, the green PE wax may be used to control melting and may be highly compatibility with other components. Green PE wax may be able to provide both internal and external lubrication (demolding effect), while also possessing high transparency and having little effect on gelling. In addition, the low volatility of Green PE wax is very important for rolling and vacuum degassing. Green PE wax, as an internal lubricant, can reduce the cohesion of polymer molecules inside the polymer, thus improving the heat generation and fluidity of plastic melt. The function of PE wax as an external lubricant improves the friction between polymer melt and the hot metal surface of the processing equipment, thereby preventing the PVC from degrading when heated and sheared.

The selection of the components of the PVC processing composition may be determined by the particular application of the composition. In some embodiments, the PVC processing compositions may be similar to known PVC processing compositions but with the replacement of a conventional lubricant for the green PE wax.

In one or more embodiments, a PVC processing composition contains 100 parts of a PVC resin. In one or more embodiments, the PVC processing composition may contain a PE wax in an amount of a range having a lower limit selected from one of 0.01, 0.1, 0.2, 0.5, 0.7, and 1.0 parts per hundred resin (PHR) and an upper limit selected from one of 0.7, 1.0, 1.2, and 1.5 PHR, where any lower limit can be used with any mathematically-compatible upper limit.

In one or more embodiments, the PVC processing composition may contain a secondary wax, which is not particularly limited. In some embodiments, the secondary wax may be a paraffin wax. In particular embodiments, the PVC processing composition may contain the secondary wax in an amount of a range having a lower limit selected from one of 0.5, 0.6, 0.7, 0.8, and 1.0 PHR and an upper limit selected from one of 1, 1.2, 1.4, and 1.5 PHR, where any lower limit can be used with any mathematically-compatible upper limit.

In one or more embodiments, the PVC processing composition may contain a fatty acid compound, which is not particularly limited. In some embodiments, the fatty acid compound may be calcium stearate. In particular embodiments, the PVC processing composition may contain the fatty acid compound in an amount of a range having a lower limit selected from one of 0.4, 0.6, 0.7, 0.8, and 1.0 PHR and an upper limit selected from one of 0.8, 1.0, 1.2, 1.4, and 1.5 PHR, where any lower limit can be used with any mathematically-compatible upper limit.

In one or more embodiments, the PVC processing composition may contain one or more tin stabilizers. The tin stabilizers are not particularly limited and may be any such tin stabilizers known to a person of ordinary skill in the art, for instance the tin stabilizers may include organotin compounds such as those derived from butyl and octyl tin oxide. In one or more embodiments, the PVC processing composition may contain the tin stabilizer in an amount of a range having a lower limit selected from one of 0.3, 0.4, 0.5, and 0.7 PHR and an upper limit selected from one of 0.5, 0.7, and 1.0 PHR, where any lower limit can be used with any mathematically-compatible upper limit.

In one or more embodiments, the PVC processing composition may contain calcium carbonate. In particular embodiments, the PVC processing composition may contain the calcium carbonate in an amount of a range having a lower limit selected from one of 1, 2, 3, 4, and 5 PHR and an upper limit selected from one of 5, 6, 7, 8, 9, and 10 PHR, where any lower limit can be used with any mathematically-compatible upper limit.

In one or more embodiments, the PVC processing composition may contain one or more UV-blocking agents, such as titanium dioxide. In particular embodiments, the PVC processing composition may contain the UV-blocking agent in an amount of a range having a lower limit selected from one of 0.5, 1.0, 1.5, and 2.0 PHR and an upper limit selected from one of 1.5, 2.0, 2.5, and 3.0 PHR, where any lower limit can be used with any mathematically-compatible upper limit.

PVC processing compositions in accordance with one or more embodiments of the present disclosure may include fillers and additives that modify various physical and chemical properties. For example, in one or more embodiments, PVC processing compositions may include processing aids in an amount of a range having a lower limit selected from one of 0.5, 1.0, 1.5, and 2.0 PHR and an upper limit selected from one of 1.5, 2.0, 2.5, and 3.0 PHR.

### EXAMPLES

Two types of HMA (hot melt adhesive) were tested: a metallocene polyethylene (mPE) based adhesive and an ethylene vinyl acetate (EVA) based adhesive. The formulation used for each adhesive is shown in Table 1 below:

TABLE 1

| | EVA based HMA | mPE based HMA |
|---|---|---|
| Metallocene polyethylene | — | 37% |
| EVA (28-400) | 35% | — |
| C9 hydrogenated | 44.7% | 37.7% |
| hydrocarbon resin | | |
| Green PE wax (GWAX 50E) | 20% | 25% |
| Antioxidant | 0.3% | 0.3% |

The green PE wax used in both formulations has the properties shown in Table 2 below.

TABLE 2

| Feature | Method | Units | Value |
|---|---|---|---|
| Dropping Point | DIN ISO 2176 | ° C. | 108 |
| Solidification Point | DIN ISO 2207 | ° C. | 94 |
| Melting/ | DIN 51007 | ° C. | 105 |
| Needle Penetration (25° C.) | DIN-51579, ASTM | 10-1 mm | 4 |
| | D1321 | | |
| Dynamic Viscosity (140° C.) | DIN EN ISO 2555 | mPas | 138 |
| Density (23° C.0 | DIN EN ISO 1183-1 | g/cm³ | 0.88 |
| Acid Value | DIN EN ISO 2114 | Mg_{(KOH)}/g | <1 |
| Saponification Value | DIN EN ISO 3681 | Mg_{(KOH)}/g | <2 |
| Yellowness Index | DIN EN ISO 11664 | — | 4 |
| Flashpoint-Clevel. | DIN EN ISO 2592 | ° C. | >250 |
| Flashpoint-Pensky M. | DIN EN ISO 2719 | ° C. | >220 |

Properties and Testing Procedures

Gardner Color—Measured by ASTM D1544. The Gardner Color scale is a single number, one-dimensional, color scale for grading the color of similarly colored liquids such as resins, vanishes, lacquers and others. Packaging hotmelt adhesives generally have initial Gardner color values of 3 or less.

Cloud Point—Measured by ASMT D 6493. This is a measure of the compatibility of the system. The lower the temperature at which the adhesive becomes cloudy, the more compatible it is. Adhesives that are cloudy even at temperatures as high as 177° C. are significantly incompatible.

Thermal Stability Testing—Measured by ASTM D4499. 150 g of each adhesive was placed in a glass jar and placed in an over set at 177° C. At 96 hours and 200 hours the samples were observed and their viscosities and color data recorded.

Viscosity—Measured by ASTM D3236 at 150 and 180° C. For the packaging industry, normally the viscosity at 177° C. is between about 700-4000 cps.

SAFT (Shear Adhesion Failure Temperature)—Measured by ASTM D 4498. SAFT values give an indication of the relative heat resistance of adhesives in the shear mode. This value is generally less important for packaging applications and often shows less variation between adhesive groups.

PAFT (Peel Adhesion Failure Temperature)—Measured by ASTM D4497. PAFT values give an indication of the relative heat resistance of adhesives in the peel mode. For general packaging applications (not frozen or refrigerated applications), the typical value is a minimum of 47° C.

Speed of Set—In order to compare large groups of adhesives for relative speed of set, it is most effective to obtain the temperature of the adhesive at which a standard Kraft paper mated to the molten adhesive exhibits fiber tear. This can be done using a temperature gradient plate. The molten adhesive is poured on the plate from hot to cool and immediately mated with Kraft paper. After equilibration, the Kraft paper is pulled away from the adhesive starting at the hot end of the gradient bar. At a certain temperature, the adhesive reaches adequate hardness to cause the Kraft paper to rip. The temperature of the adhesive at this point is recorded. The higher the temperature at which this occurs, the faster setting the adhesive.

Bond Testing—Bonds were made in using the test adhesives on Adherent Laboratories standard substrate, corrugated board from Rock Tenn. Each adhesive was applied at 177° C. in approximately a 2-3 mm wide bead (uncompressed) to the primary substrate, then compressing the bond with the secondary substrate then allowed to stand at room temperature for at least 12 hours. The finished bonds were then placed at −18, 3 and 23° C. for at least 24 hours before being tom apart at the conditioning temperature. The resulting torn bond was evaluated for the percentage of adhesive area that is covered with paper fiber (% fiber tear). A minimum of 5 bonds were evaluated for each adhesive/condition combination. The average of the % Fiber tear is recorded as described according to Bond result categories:
Excellent=100-80% fiber tear
Good=50-79% fiber tear
Marginal=20-49% fiber tear
Fail=0-19% fiber tear

IoPP Testing (Institute of Packaging Professionals)—Measured by IOPP Test. This test method determines relative heat failure of an adhesive to corrugated in the “cleavage” mode. Cleavage failure mode is a combination of peel and shear modes that was designed to more closely mimic the actual forces applied to a case closure adhesive during transit. The temperature recorded is the highest temperature that the bonds remained intact.

Results

Table 3 below shows the results obtained for each property on the two formulations tested for HMA (hotmelt adhesive).

TABLE 3

| | EVA-based HMA | mPE based HMA |
|---|---|---|
| Viscosity at 180° C. (cps) | 3660 | 1218 |
| Viscosity at 150° C. (cps) | 13520 | 7675 |
| Molten Gardner color | 1 | 1 |
| Cloud point (° C.) | 160 | 96 |
| PAFT (° C.) | 49 | 54 |
| SAFT (° C.) | 72 | 83 |
| Speed of set comparison (gradient | 65 | 69 |
| bar, temp at FT) (° C.) | | |
| Bond testing (corrugated substrate) | | |
| (Eval. Fiber Tear) | | |
| Bonds at −18° C. | Excellent | Excellent |
| Bonds at 4° C. | Excellent | Excellent |
| Bonds at RT | Excellent | Excellent |
| Bonds at 60° C. | Marginal | Marginal |
| Specific Gravity | 0.955 | 0.925 |
| IOPP Heat resistance (° C.) | 35 | 35 |
| Thermal Stability (200 hrs at 177° | | |
| C.) | | |
| Initial viscosity at 180° C. (cps) | 3660 | 1218 |
| Initial Garder color | 1 | 1 |
| 200 hr viscosityat 180° C. (cps) | 5960 | 1313 |
| 200 hour Gardner colo | 8 | 10 |

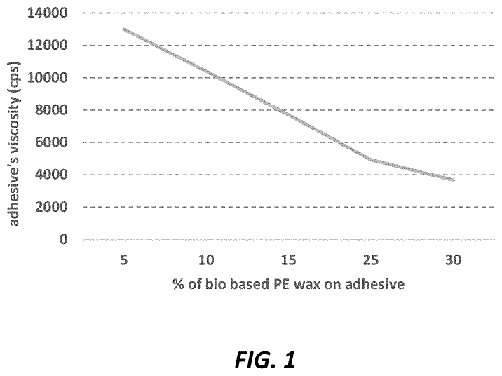

A Viscosity curve was also tested for metallocene PE based adhesive, varying the percentage of green PE wax on the adhesive. The result is shown in FIG. 1 and shows the change on rheology behavior of the adhesive as a function of wax amount added.

The tests performed showed that the green PE wax described herein is suitable for usage in hotmelt adhesive formulations. Different range of raw materials can be optimized to improve compatibility performance depending on the adhesive's usage.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. A composition, comprising:
a polyethylene wax, in which the polyethylene comprises at least a portion of ethylene that is obtained from a renewable source of carbon.

2. The composition of claim 1, wherein the composition contains the polyethylene wax in an amount in the range of 0.5 to 65% by weight (wt. %).

3. The composition of claim 1, wherein the polyethylene wax exhibits a biobased carbon content as determined by ASTM D6866 of at least 10%.

4. The composition of claim 1, wherein the composition exhibits a biobased carbon content as determined by ASTM D6866 of at least 5%.

5. The composition of claim 1, wherein the composition comprises:
a polyolefin resin in an amount of 25 to 45 wt. %;
a tackifier in an amount of 30 to 55 wt. %;
a second wax in an amount of 1 to 20 wt. %; and
an antioxidant in an amount of 0.1 to 2 wt. %.

6. The composition of claim 5, wherein the polyolefin resin is a EVA copolymer.

7. The composition of claim 6, wherein the composition comprises:
an EVA copolymer in an amount of 25 to 35 wt. %;
a tackifier in an amount of 2 to 10 wt. %;
a second polyolefin in an amount of 3 to 10 wt. %;
a stabilizer in an amount of 0.1 to 2 wt. %; and
an antioxidant in an amount of 0.1 to 2 wt. %.

8. The composition of claim 9, wherein the composition comprises:
calcium carbonate in an amount of 50 to 85 wt. %;
a coupling agent in an amount of 1 to 10 wt. %; and
an additive in an amount of 0.1 to 30 wt. %.

9. The composition of claim 1, wherein the composition comprises:
a pigment in an amount of 10 to 60 wt. %;
a second wax in an amount of 0.1 to 3 wt. %;
a binder in an amount of 20 to 80 wt. %; and
an ink oil in an amount of 5 to 10 wt. %.

10. The composition of claim 1, wherein the composition comprises:
a pigment in an amount of 10 to 60 wt. %;
a binder in an amount of 20 to 80 wt. %; and
a matte vehicle in an amount of 1 to 10 wt. %; and
an ink oil in an amount of 5 to 10 wt. %.

11. The composition of claim 1, wherein the composition comprises:
PVC in an amount of 100 parts by mass;
a stabilizer in an amount of 0.3 to 1 part by mass;
a second wax in an amount of 0.6 to 1.5 parts by mass; and
an additive in an amount of 0.1 to 5 parts by mass.

12. The composition of claim 17, wherein the composition comprises:
the green PE wax in an amount ranging from 3 to 20 wt %;
a liquid component in an amount ranging from 1 to 80 wt %;
a skin conditioning agent and/or emollient in an amount ranging from 2 to 90 wt %;
optionally, a surfactant in an amount up to 8 wt %; and
optionally, a colorant in an amount up to 30 wt %.

13. An article comprising a substrate and a coating on the substrate, the coating comprising the composition of claim 1.

14. The article of claim 13, wherein the composition is a hot melt adhesive and also comprises a polyolefin resin, a tackifier, and a second wax.

15. The article of claim 13, wherein the composition is an ink and also comprises pigment, binder, an ink oil, and optionally a second wax or matte vehicle.

16. An article comprising the composition of claim 8, wherein the composition is a filler masterbatch.

17. A process for producing a composition that comprises a polyethylene wax, the method comprising:
combining the polyethylene wax with at least one other component to form the composition of claim 1.

18. The process of claim 17, further comprising:
polymerizing ethylene at least partially obtained from a renewable source of carbon to produce the polyethylene wax.

19. The process of claim 17, the method further comprising:
fermenting a renewable source of carbon to produce ethanol;
dehydrating the ethanol to produce ethylene;
polymerizing the ethylene to produce the polyethylene wax.

20. The process of claim 19, wherein the renewable source of carbon is at least one plant material selected from the group consisting of sugar cane and sugar beet, maple, date palm, sugar palm, sorghum, American agave, corn, wheat, barley, sorghum, rice, potato, cassava, sweet potato, algae, fruit, materials comprising cellulose, wine, materials comprising hemicelluloses, materials comprising lignin, wood, straw, sugarcane bagasse, sugarcane leaves, corn stover, wood residues, paper, and combinations thereof.
